(19) United States
(12) Patent Application Publication
(10) Pub. No.: US 2024/0285636 A1
(43) Pub. Date: Aug. 29, 2024

(54) PHARMACEUTICAL COMPOSITION AND USE OF THE PHARMACEUTICAL COMPOSITION

(71) Applicant: LIBBS FARMACÊUTICA LTDA, São Paulo (BR)

(72) Inventors: Tiago da Cunha SAIS, São Paulo (BR); Adriana Lara SANTOS, São Paulo (BR); Cassio Yooiti YAMAKAWA, São Paulo (BR); Aurora Emi Tanabe MIURA, São Paulo (BR); Simone Mayumi KONDO, São Paulo (BR); Maiely Bueno MILARÉ, São Paulo (BR)

(73) Assignee: LIBBS FARMACÊUTICA LTDA, São Paulo (BR)

(21) Appl. No.: 18/569,621

(22) PCT Filed: Mar. 16, 2022
(86) PCT No.: PCT/BR2022/050090

§ 371 Date: Dec. 13, 2023

(30) Foreign Application Priority Data
Jun. 14, 2021 (BR) 10 2021 011533-5

(51) Int. Cl. A61K 31/519; A61K 9/20

(52) U.S. Cl. CPC A61K 31/519; A61K 9/2009; A61K 9/2018; A61K 9/2027; A61K 9/2031; A61K 9/2054

(57) ABSTRACT

The present invention relates to new pharmaceutical compositions comprising {1S-[1α,2α,3β(1S*,2R*),5β}-3-(7-{[2-(3,4-difluorophenyl)-cyclopropyl]amino}-5-(propylthio)-3H-1,2,3-triazol[4,5-d]pyrimidin-3-yl)-5-(2-hydroxyethoxy) cyclopentane-1,2-diol, commercially known as ticagrelor, and the copolymer formed by polyvinyl alcohol units and polyethylene glycol units as a binder.

### FIELD OF THE INVENTION

The present invention relates to new pharmaceutical compositions comprising {1S-[1α,2α,3β(IS*,2R*),5β}-3-(7-{[2-(3,4-difluorophenyl)-cyclopropyl]amino}-5-(propylthio)-3H-1,2,3-triazol[4,5-d]pyrimidin-3-yl)-5-(2-hydroxyethoxy) cyclopentane-1,2-diol, commercially known as ticagrelor, and the copolymer formed by polyvinyl alcohol units and polyethylene glycol units as a binder.

The composition according to the present invention has superior stability and dissolution compared to known pharmaceutical compositions for ticagrelor.

### BACKGROUND OF THE INVENTION

O compound {1S-[1α,2α,3β(1S*,2R*),5β}-3-(7-{[2-(3,4-difluorophenyl)-cyclopropyl]amino}-5-(propylthio)-3H-1,2,3-triazol[4,5-d]pyrimidin-3-yl)-5-(2-hydroxyethoxy) cyclopentane-1,2-diol, known as ticagrelor, is an antiplatelet agent cyclopentyl-triazole-pyrimidine, oral antagonist of the P2Y12 receptor, to which it binds reversibly.

### Chemical Structure of Ticagrelor

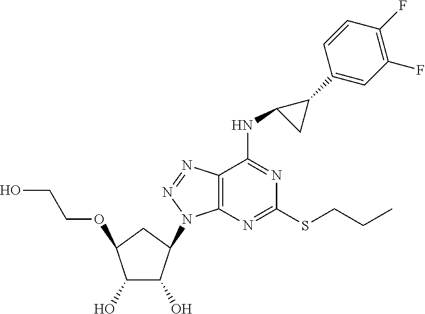 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

This active ingredient is prescribed for the prevention of atherothrombotic events in adults with acute coronary syndromes (ACS), for example unstable angina pectoris, myocardial infarction, including those treated medically or with percutaneous coronary intervention or coronary artery bypass graft (CABG).

Ticagrelor provides selective and reversible inhibition of adenosine diphosphate-induced platelet aggregation, with action onset and offset faster than other medicaments for the same purpose, for example clopidogrel.

Despite its importance in antiplatelet therapy, ticagrelor is a molecule that presents difficulties in formulation and handling.

Ticagrelor is an active ingredient that is difficult to dissolve, being classified as a class IV medicament in the Biopharmaceutical Classification System. Said System divides medicaments into 4 classes, taking into account their solubility, permeability and absorption. A class I medicaments has high solubility and high permeability, having good absorption, while Class IV medicaments have low solubility and low permeability, whose speed and degree of absorption must be considered on a case-by-case basis.

In this sense, it is a problem of the prior art to provide pharmaceutical compositions comprising ticagrelor exhibiting improved stability and dissolution.

Several prior art documents address pharmaceutical compositions of ticagrelor without, however, resolving the problems mentioned above.

Particularly, patent application WO2011076749 refers to formulations comprising the active ingredient ticagrelor with a particle size ranging between 1 μm and 150 μm. However, this document does not disclose a pharmaceutical composition comprising ticagrelor and the excipients of the present invention. To solve problems related to the bioavailability of ticagrelor, this document studies different particle sizes of the active ticagrelor, solving it through a specific particle size. No particular effects are associated with the excipients of the compositions of this document, which are distinct from those used in the present invention.

The patent PI 0715712-6 addresses problems related to the bioavailability of ticagrelor, claiming that existing compositions retained some of this active ingredient, consequently reducing its bioavailability. This document addresses this issue through the combination of specific excipients, particularly the combination of the active ingredient ticagrelor with the excipients mannitol, calcium phosphate, hydroxypropyl cellulose and sodium starch glycolate (as in example 1). It should be noted that the excipients in the patent PI 0715712-6 are different from those used in the present invention.

Document PI 0111328-3 discloses formulations of ticagrelor, and seeks to address problems related to substances in amorphous form, such as stability and difficulty of formulation, particularly addressing the obtaining of crystalline and amorphous forms of ticagrelor. It is worth noting that the excipients of the formulations embodied in this document are lactose, croscarmellose sodium, corn starch and magnesium stearate, with or without polyvinylpyrrolidone. Therefore, this document does not disclose the pharmaceutical composition of the present invention, as well as the technical problem addressed by it.

In order to solve the problems of the art, the inventors of the present invention have surprisingly managed to overcome the deficiencies of the prior art by means of pharmaceutical compositions comprising ticagrelor and copolymer formed by polyvinyl alcohol units and polyethylene glycol units as a binder.

In particular, none of the prior art documents mention the use of a copolymer formed by polyvinyl alcohol units and polyethylene glycol units as a binder in ticagrelor formulations, and are silent on the effects of this excipient in improving the dissolution of the active ingredient, consequently affecting its stability.

Furthermore, the use of the copolymer formed by polyvinyl alcohol units and polyethylene glycol units as a binder in ticagrelor formulations prevents the formation of degradation products, contributing to its stability.

No prior art document discloses or suggests compositions as described in the present patent application to overcome issues related to the stability and solubility of ticagrelor pharmaceutical compositions.

### SUMMARY OF THE INVENTION

In one embodiment, the present invention relates to a pharmaceutical composition comprising ticagrelor and copolymer formed by polyvinyl alcohol units and polyethylene glycol units as a binder.

In a preferred embodiment, the pharmaceutical composition of the present invention may further comprise additional excipients selected from diluents, disintegrants, lubricants, glidants and, optionally one or more pharmaceutically acceptable coating ingredients, and/or purified water.

In a preferred embodiment, the pharmaceutical composition according to the present invention comprises ticagrelor and copolymer formed by polyvinyl alcohol units and polyethylene glycol units as a binder, and the diluents mannitol and dibasic calcium phosphate dihydrate, the glidant colloidal silicon dioxide and pharmaceutically acceptable lubricants, and optionally the disintegrant croscarmellose sodium. Particularly, the lubricants can be selected from magnesium stearate and/or sodium stearyl fumarate.

Additionally, the present application also relates to the use of said compositions for the manufacture of an antiplatelet medicament.

### BRIEF DESCRIPTION OF FIGURES

FIGS. 1 and 2 refer to the dissolution profile results of the tests carried out in Examples 1 and 3.

### DETAILED DESCRIPTION OF THE INVENTION

The present invention relates to new pharmaceutical compositions comprising 1S-[1α,2α,3β(1S*,2R*),5β}-3-(7-{[2-(3,4-difluorophenyl)-cyclopropyl]amino}-5-(propylthio)-3H-1,2,3-triazol[4,5-d]pyrimidin-3-yl)-5-(2-hydroxyethoxy) cyclopentane-1,2-diol (ticagrelor) and copolymer formed by polyvinyl alcohol units and polyethylene glycol units as a binder.

Preferably, ticagrelor is present in a range of 25.0 to 35.0% by weight, with respect to the total weight of the composition, and the copolymer formed by polyvinyl alcohol units and polyethylene glycol units is present in a range of 3.0 to 8.0% by weight, with respect to the total weight of the composition.

In a preferred embodiment, the pharmaceutical composition of the present invention additionally comprises:
from 32.0 to 45.0% by weight, with respect to the total weight of the composition, of the diluent mannitol;
from 15.0 to 25.0% by weight, with respect to the total weight of the composition, of the diluent dibasic calcium phosphate dihydrate;
from 0.5 to 2.5% by weight, with respect to the total weight of the composition, of the glidant colloidal silicon dioxide;
from 0.5 to 2.5% by weight, with respect to the total weight of the composition, of one or more pharmaceutically acceptable lubricants, preferably selected from sodium stearyl fumarate and/or magnesium stearate.
optionally, one or more coating ingredients, and
optionally, purified water.

In another embodiment, the pharmaceutical composition of the present invention further comprises:
from 32.0 to 45.0% by weight, with respect to the total weight of the composition, of diluent mannitol;
from 15.0 to 25.0% by weight, with respect to the total weight of the composition, of the diluent dibasic calcium phosphate dihydrate;
from 2.0 to 5.0% by weight, with respect to the total weight of the composition, of the disintegrant croscarmellose sodium;
from 0.5 to 1.5% by weight, with respect to the total weight of the composition, of the glidant colloidal silicon dioxide;
from 0.5 to 2.5% by weight, with respect to the total weight of the composition, of one or more lubricants, selected from sodium stearyl fumarate and/or magnesium stearate,
optionally, one or more coating ingredients, and
optionally, purified water.

The possible one or more pharmaceutically acceptable coating ingredients according to the present invention can be chosen from those available in the prior art. A person skilled in the art would be able to choose the appropriate amount of these pharmaceutically acceptable coating ingredients based on the desired final composition.

The pharmaceutical compositions according to the present invention can be used in the manufacture of anticoagulant medicaments.

### Process for Manufacturing the Composition

The pharmaceutical compositions of the present invention can be obtained by means of processes known in the art, for example in a fluidized bed or in a high shear mixer, and a person skilled in the art would be fully capable of selecting the most appropriate process according to its interests and available equipment.

A non-limiting example of a suitable process for manufacturing the pharmaceutical composition of the present invention, in the pharmaceutical form of a coated tablet, comprises the steps of:
Step 1) Preparation of the binder solution: solubilizing the copolymer formed by polyvinyl alcohol units and polyethylene glycol units in purified water and reserve it.
Step 2) Mix: mixing, in a BIN type mixer, mannitol, colloidal silicon dioxide, ticagrelor and dibasic calcium phosphate dihydrate.
Step 3) Granulation: transferring the mixture from Step 2 to the fluidized bed or to the high shear mixer and carry out granulation with the binder solution from Step 1, and drying the granulate.
Step 4) Final mix: classifying the granulate and mixing in a BIN type mixer with croscarmellose sodium and lubricant (vegetable magnesium stearate or sodium stearyl fumarate).
Step 5) Compression: compressing the mixture obtained in Step 4 into a 9 mm circular punch, with an average weight of 300 mg.
Step 6) Coating: preparing the coating suspension by dispersing Light Green Opadry in purified water, and coating the tablets with the suspension obtained in Step 5.

### EXAMPLES

Initially, it should be noted that the pharmaceutical compositions according to the present invention will be described below according to the particular, but not limiting, embodiments, since their embodiments may be carried out in different forms and variations and depending on the application desired by the person skilled in the art, with the scope of the invention being defined according to the claims.

### Example 1—Dissolution Profile

Pharmaceutical compositions comprising ticagrelor in accordance with the teachings of the prior art can be found in formulations A, B and C below, and a pharmaceutical composition according to the present invention can be found in formulation D.

Formulation A was produced in accordance with the teachings of the patent Pd 0715712-6.

TABLE 1
Composition ingredients

| | | A | B | C | D |
|---|---|---|---|---|---|
| Function | Ingredient | % | % | % | % |
| Active | ticagrelor | 30.0 | 30.0 | 30.0 | 30.0 |
| Diluent | mannitol | 42.0 | 41.0 | 41.0 | 41.0 |
| | dibasic calcium | 21.0 | 21.0 | 21.0 | 21.0 |
| | phosphate | | | | |
| | dihydrate | | | | |
| Binder | Hyprolosis | 3.0 | — | — | — |
| | Povidone | — | 3.0 | — | — |
| | Carbopol | — | — | 3.0 | — |
| | copolymer formed | — | — | — | 3.0 |
| | by polyvinyl alcohol | | | | |
| | units and | | | | |
| | polyethylene glycol | | | | |
| | units | | | | |
| Disintegrant | Sodium starch | 3.0 | — | — | — |
| | glycolate | | | | |
| | Croscarmellose | — | 3.0 | 3.0 | 3.0 |
| | sodium | | | | |
| Lubricant | Sodium Stearyl | — | 1.0 | 1.0 | 1.0 |
| | Fumarate | | | | |
| | Magnesium | 1.0 | — | — | — |
| | stearate | | | | |
| Glidant | Colloidal silicon | — | 1.0 | 1.0 | 1.0 |
| | dioxide | | | | |
| Coating film | Opadry light green | 4.0 | 3.5 | 3.5 | 3.5 |
| | 03B81004 | | | | |

To evaluate the dissolution of the composition of the present invention (Formulation D), a comparative test was carried out with those of the prior art according to formulations A, B and C, and also with the only medicament available on the market containing the active substance, Brilinta®.

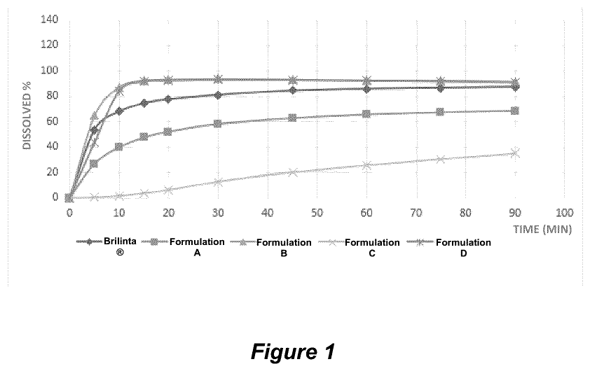

As can be seen in FIG. 1, the dissolution profile of the pharmaceutical composition according to the present invention, formulation D, is the profile that most closely resembles the dissolution profile of the medicament that is commercially available, Brilinta®.

This is advantageous when one wants to manufacture a product that is similar to a registered medicament and that must have equivalent properties in terms of bioavailability and dissolution profile.

### Example 2—Stress Tests

A stress test was carried out with the ticagrelor compositions according to Example 1 above, with formulations A, B and C in accordance with the state of the art and formulation D in accordance with the pharmaceutical composition of the present invention.

The stress test is a forced degradation test and consisted of incubating tablet samples for 14 days in vials exposed to the two conditions below:
(1) wet thermal condition: open bottle is incubated at a temperature of 70° C. and 75% relative humidity.
(2) dry thermal condition: closed bottle is incubated at a temperature of 70° C. and 75% relative humidity.

These conditions employed are the most realistic conditions, considering the actual degradation profile of the product in stability.

The content of the active compound (percentage of ticagrelor in the formulation) is measured before the start of the experiment (initial).

The following tables present the results for each of the pharmaceutical compositions A to D.

TABLE 2
Results for formulation A (state of the art)
Formulation A

| | | |
|---|---|---|
| Initial | Dry thermal - 14 days | Wet thermal - 14 days |

Content (%)

| | | |
|---|---|---|
| 93.5 | 93.3 | 93.6 |

TABLE 3
Results for formulation B (state of the art)
Formulation B

| | | |
|---|---|---|
| Initial | Dry thermal - 14 days | Wet thermal - 14 days |

Content (%)

| | | |
|---|---|---|
| 100.2 | 99.5 | 100.05 |

TABLE 4
Results for formulation C (state of the art)
Formulation C

| | | |
|---|---|---|
| Initial | Dry thermal - 14 days | Wet thermal - 14 days |

Content (%)

| | | |
|---|---|---|
| 95.5 | 100.7 | 100.8 |

TABLE 5
Results for formulation D (inventive)
Formulation D

| | | |
|---|---|---|
| Initial | Dry thermal - 14 days | Wet thermal - 14 days |

Content (%)

| | | |
|---|---|---|
| 98.6 | 102.2 | 104.6 |

It is noted that formulation D, according to the pharmaceutical composition of the present invention, presented an excellent initial active substance content (after manufacturing), superior to formulation A according to the patent PI 0715712-6. Furthermore, it is noteworthy that formulation D according to the pharmaceutical composition of the present invention remained stable after 14 days of thermal stress, both in wet and dry conditions.

### Example 3—Stability

To ascertain the stability of the pharmaceutical composition according to the present invention, formulation E below was prepared:

TABLE 6
Formulation E (inventive)

| Function | Ingredient | % |
|---|---|---|
| Active | ticagrelor | 30.0 |
| Diluent | mannitol | 38.0 |
| | dibasic calcium phosphate dihydrate | 21.0 |
| Binder | copolymer formed by polyvinyl alcohol | 5.0 |
| | units and polyethylene glycol units | |
| Disintegrant | Croscarmellose sodium | 3.0 |
| Lubricant | Magnesium stearate | 2.0 |
| Glidant | Colloidal silicon dioxide | 1.0 |
| Coating film | Opadry light green 03B81004 | 3.5 |

The tablets comprising the pharmaceutical composition according to the present invention were incubated at 40° C. and 75% relative humidity (RH). The amount of ticagrelor present in the sample (content) was measured before the beginning of this study and after 6 months.

Furthermore, a second test was carried out with incubation at 30° C. and 75% relative humidity. The active substance content was measured after 12 months.

The same procedure was performed with the ticagrelor reference product, Brilinta® (state of the art), incubated under the same conditions as above. The results obtained are shown in table 7:

TABLE 7
stability study results - content

| | Formulation E | State of the art |
|---|---|---|
| Active substance content | (inventive) | formulation |
| Initial | 99.80% | 98.85% |
| After 6 months 40° C./75% RH | 97.24% | 98.93% |
| After 12 months 30° C./75% RH | 98.9% | 98.55% |

These results demonstrate that the formulation of the invention is stable, in terms of the active substance content parameter, contrary to what is suggested by the state of the art.

Moreover, it was carried out a comparative study of the dissolution profile between the formulation of the present invention E and the medicament Brilinta®, carried out before and after 6 months.

Table 8 below shows a comparison of the stability of the pharmaceutical composition E (inventive) and the composition according to the prior art medicament (method: 0.05 M acetate buffer medium, pH 4.5, Tween 80 0.08%, apparatus 1 (40 mesh basket), with rotation of 100 rpm), at the beginning and after 6 months of incubation at a temperature of approximately 40° C. and a relative humidity of approximately 75%.

TABLE 8
dissolution curve - amount dissolved over time

| | | State of the art | | |
|---|---|---|---|---|
| | Formulation of | formulation | | Formulation E |
| Time | the state of | (after 6 months | Formulation E | (after 6 months |
| (minutes) | the art (initial) | 40° C./75% RH) | (initial) | 40° C./75% RH) |
| 0 | 0 | 0 | 0 | 0 |
| 5 | 36.0 | 1.8 | 19.3 | 11.6 |
| 10 | 53.2 | 20.0 | 57.4 | 39.5 |
| 15 | 59.4 | 43.4 | 70.3 | 61.3 |
| 20 | 64.4 | 53.4 | 75.5 | 73.0 |
| 30 | 69.6 | 63.4 | 82.7 | 79.1 |
| 45 | 74.9 | 71.3 | 88.8 | 88.5 |
| 60 | 77.8 | 75.2 | 91.9 | 92.1 |

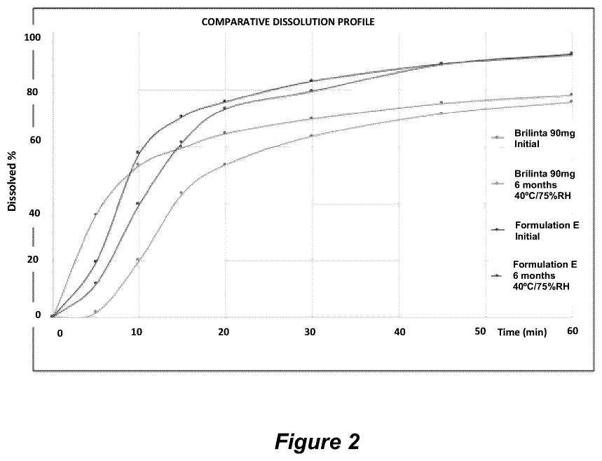

These results are graphically represented in FIG. 2. It is noted that the dissolution profile of formulation E (according to the present invention) did not change after 6 months, unlike Brilinta® (state of the art). The percentage dissolved after 60 minutes is the same in the initial condition and after 6 months for the inventive composition (FIG. 2, red lines), but there is a clear decline in this percentage for the reference medicament, Brilinta®, after 6 months (blue lines).

The drop in the dissolution profile demonstrates that the pharmaceutical composition according to the present invention is more stable than the formulation according to the state of the art. Furthermore, the amount dissolved after 60 minutes is higher than the percentage achieved by the state of the art reference composition.

1-9. (canceled)

10. A pharmaceutical composition comprising:
(a) {1S-[1α,2α,3β(IS*,2R*),5β}-3-(7-{[2-(3,4-difluorophenyl)-cyclopropyl]amino}-5-(propylthio)-3H-1,2,3-triazol[4,5-d]pyrimidin-3-yl)-5-(2-hydroxyethoxy) cyclopentane-1,2-diol; and
(b) a copolymer formed by polyvinyl alcohol units and polyethylene glycol units as a binder.

11. The composition of claim 10 comprising from 25.0 to 35.0% by weight of (a), based on a total weight of the composition.

12. The composition of claim 10 comprising from 3.0 to 8.0% by weight of (b), based on a total weight of the composition.

13. The composition of claim 10, further comprising one or more additional excipients selected from a diluent, a pharmaceutically acceptable lubricant, a glidant, a pharmaceutically acceptable coating ingredient, purified water, a disintegrants, or mixtures thereof.

14. The composition of claim 10, further comprising:
(c) from 32.0 to 45.0% by weight of mannitol;
(d) from 15.0 to 25.0% by weight of dibasic calcium phosphate dihydrate;
(e) from 0.5 to 2.5% by weight of colloidal silicon dioxide;
(f) from 0.5 to 2.5% by weight of one or more pharmaceutically acceptable lubricants;
(g) optionally, one or more coating ingredients, and
(h) optionally, purified water
wherein all percentages by weight are based on a total weight of the composition.

15. The composition of claim 13 comprising the pharmaceutically acceptable lubricant, wherein the pharmaceutically acceptable lubricant is selected from sodium stearyl fumarate or magnesium stearate.

16. The composition of claim 13 comprising the disintegrant, wherein the disintegrant is croscarmellose sodium.

17. The composition of claim 16, wherein the croscarmellose sodium is present in an amount from 2.0 to 5.0% by weight, based on a total weight of the composition.

18. A method for inhibiting adenosine diphosphate-induced platelet aggregation comprising administering the composition of claim 10 to an individual in need thereof.
